SEQUENCE LISTING (3 sequences)

SEQ ID NO: 1 - RNA, 22 nt, Homo sapiens
  misc_RNA (1)..(22)
uagcuuaucagacugauguuga

SEQ ID NO: 2 - RNA, 23 nt, Homo sapiens
  misc_RNA (1)..(23)
agcuacauugucugcuggguuuc

SEQ ID NO: 3 - RNA, 22 nt, Homo sapiens
  misc_RNA (1)..(22)
uagcagcacguaaauauuggcg

(19) United States
(12) Patent Application Publication
(10) Pub. No.: US 2012/0196293 A1
(43) Pub. Date: Aug. 2, 2012

(54) METHOD AND KIT FOR IN VITRO DIAGNOSIS OF ATHEROSCLEROSIS

(75) Inventors: Suh-Hang Hank Juo, Kaohsiung City (TW); Yung-Song Wang, Kaohsiung City (TW); Pei-Chien Tsai, Kaohsiung City (TW)

(73) Assignee: KAOHSIUNG MEDICAL UNIVERSITY, Kaohsiung City (TW)

(21) Appl. No.: 13/334,798

(22) Filed: Dec. 22, 2011

(30) Foreign Application Priority Data
Jan. 28, 2011 (TW) 100103331

(51) Int. Cl. C12Q 1/68

(52) U.S. Cl. 435/6.12

(57) ABSTRACT

A method for in vitro diagnosis of atherosclerosis, comprising: (a) obtaining a sample from a subject; (b) determining expression levels of one or more microRNAs (miRNAs) as atherosclerotic biomarkers and an internal control RNA; (c) computing the relative expression levels of the one or more miRNAs as atherosclerotic biomarkers; (d) computing a prediction model with one or more variables, wherein the variable includes one or more relative expression levels of the one or more miRNAs as atherosclerotic biomarkers and one or more risk factors of atherosclerosis; and (e) computing a prediction probability by the prediction model, wherein the subject is diagnosed with atherosclerosis if the probability is more than 0.5 is presented. A kit for in vitro diagnosis of atherosclerosis or prognosis of atherosclerosis-inducing diseases is also presented.

### FIELD OF THE INVENTION

The present invention relates to a method and a kit for in vitro diagnosis and/or prediction of disease. More specifically the invention relates to a method and a kit for in vitro diagnosis and/or prediction of diseases caused by atherosclerosis.

### BACKGROUND OF THE INVENTION

Atherosclerosis is a complicated vascular disease and also a major pathological factor which causes myocardial infarction and stroke. Atherosclerosis can be caused by lipid accumulation in the artery wall, which thickens arterial intima-media and often leads the obstruction of artery. This process often occurs in the aorta, coronary artery, cerebral artery, renal arteries, etc. Atherosclerosis is one leading cause of morbidity and mortality in most developed countries, and has been expected to be the leading cause of death worldwide by 2020. The pathogenic process of atherosclerosis is a slow and complex process, which may be divided into three stages: the first stage is the formation of a fatty streak, the second stage is the formation of an advanced fibrous plaque, and the third stage is the formation of a complicated lesion.

The conventional diagnostic tools for atherosclerosis are ultrasound system and angiography, which are used to examine the lesions of arterial stenosis and calcification. However, the conventional diagnostic methods are only suitable for the cases that the atherosclerosis lesions are clearly visible. In addition, the risk factors of atherosclerosis, such as age, gender, blood pressure, fasting blood glucose, total cholesterol, triglyceride and the like are also used to help in diagnosing or predicting the risk of disease for atherosclerosis and its related diseases. But the sensitivity and specificity of these diagnosis or prediction tools need to be improved. Consequently, it is actually necessary to develop a new tool and a kit to improve early prediction and detection of atherosclerosis, and also predict the risk of diseases caused by atherosclerosis.

According to the previous studies, microRNAs (miRNAs) are important regulators for cell growth, differentiation and apoptosis, which regulate gene expression (Costinean S, et al. Proc Natl Acad Sci USA. 2006; 103: 7024-7029, Ambros V. Nature. 2004; 431: 350-355 and Hwang H W, et al. Br J Cancer 2006; 94: 776-780). Therefore, miRNA plays an important role in the cell growth and physiology. Consequently, dysregulation of the miRNA function may lead to human diseases. Because the cell dedifferentiation, growth and apoptosis are important pathological alterations in carcinogenesis, many studies focused on the role of the miRNA in the carcinogenesis process. Currently, it is believed that miRNAs may play roles as tumor suppressors or oncogenes (Esquelq-Kerscher A, et al. Nature Reviews Cancer. 2006; 6: 259-269). In addition, some studies have demonstrated the expression of the miRNA in the mouse cardiovascular system (Lagos-Quintana M, et al. Curr Biol. 2002; 12: 735-739; Van Rooij E, et al. Proc Natl Acad Sci USA. 2006; 103: 18255-18260). However, the role of the miRNA in the atherosclerotic disease is still needed to be investigated.

### BRIEF DESCRIPTION OF THE DRAWINGS

The invention can be more fully understood by reading the subsequent detailed description and examples with references made to the accompanying drawing, wherein:

FIG. 1 shows the gene encoding the pre-miRNA-21 is located at the chromosome 17q23.1 (A). The stem-loop structure of the pre-miRNA-21 is also shown (B), wherein the mature miRNA-21 (miR-21) is underlined by the black line. The gene encoding the pre-miRNA-221 is located at the chromosome Xp11.3 (C). The stem-loop structure of the pre-miRNA-221 is shown in (D), wherein the mature miRNA-221 (miR-221) is underlined by the black line.

FIG. 2 shows serum miR-21 and miR-221 levels (log 2^{-ΔCt}) of healthy controls (H), atherosclerosis subjects (A) and stroke patients (S) for all study subjects

FIG. 3 shows receiver-operating characteristic (ROC) curves of different models (^{†} Traditional risk factors: age, sex, ever smoking, diabetes, hypertension and hyperlipidemia; ^{‡} the diagonal line represented the reference line with area under curve (AUC)=0.5; *significant difference (p<0.05) of AUCs between model 4 and the other three models calculated by Wilcoxon-Mann-Whitney U test purposed by Delong et al (p=0.005 for models 4 vs. 1, 0.007 for models 4 vs. 2, and 0.023 for models 4 vs. 3).

### SUMMARY OF THE INVENTION

The present invention provides a method for in vitro diagnosis of atherosclerosis, comprising: (a) obtaining a sample from a subject; (b) determining expression levels of one or more miRNAs (microRNAs) as atherosclerotic biomarkers and an internal control RNA; (c) computing relative expression levels of the one or more miRNAs as atherosclerotic biomarkers; (d) computing a prediction model with one or more variables, wherein the variables include relative expression levels of the one or more miRNAs as atherosclerotic biomarkers and one or more risk factors for atherosclerosis; and (e) computing a disease risk probability by the prediction model, wherein the subject is diagnosed with atherosclerosis if the disease risk probability is more than 0.5.

The present invention also provides a kit for in vitro diagnosing atherosclerosis and/or predicting the diseases caused by atherosclerosis.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

As used herein, the term “a” or “an” is employed to describe elements and components of the invention. This is done merely for convenience and to give a general sense of the invention. This description should be read to include one or at least one and the singular also includes the plural unless it is obvious that it is meant otherwise.

As used herein, the term “or” is employed to describe “and/or”.

The present invention discloses a method for selecting a miRNA for use as a disease diagnosis biomarker, in order to obtain the miRNA for diagnosing atherosclerosis in vitro. The method for selecting a miRNA for use as a disease diagnosis biomarker comprises the following steps of: (a) obtaining samples from subjects, wherein the subjects are composed of people suffering from the disease and people not suffering from the disease; (b) determining expression levels of candidate miRNAs and an internal control RNA in the samples; (c) computing relative expression levels of the candidate miRNAs; (d) computing a prediction model with one or more variables, wherein the variables include relative expression levels of one or more candidate miRNAs and one or more risk factors of the disease; and (e) computing a disease risk probability, sensitivity and specificity by the prediction model; wherein the one or more candidate miRNAs with the highest sensitivity and the highest specificity.

In one embodiment, the disease is atherosclerosis or disease caused by atherosclerosis, such as stroke, myocardial infarction, peripheral arterial occlusion disease and acute coronary syndrome. The risk factors of the disease are risk factors of atherosclerosis.

The technical term “microRNA (which is referred to as a miRNA)” recited herein is defined as a single-stranded RNA molecule of about 21-23 nucleotides in length. The miRNA is first transcribed as a pri-miRNA with a cap and a poly-A tail, and then processed and shortened into a 70-nucleotide stem-loop structure known as pre-miRNA. Afterwards, the pre-microRNA is processed to a mature miRNA. The mature miRNA is complementary to a part of one or more mRNAs. Animal miRNAs are usually complementary to a site in a 3′ untranslated region (which is referred to as an UTR). The annealing of the miRNA to the mRNA triggers the regulation of gene expression by suppressing protein translation or by promoting mRNA degradation.

In one embodiment, the samples include blood, tissue and body fluid. In another embodiment, the sample is blood or tissue.

In one embodiment, the expression levels of the candidate miRNAs and the internal control RNA are determined by (but not limited to) a northern blot hybridization, an RNase protection assay, or a quantitative real-time RT-PCR. Wherein the technical term “quantitative real-time reverse transcription polymerase chain reaction (which is referred to as a qRT-PCR)” is defined as a method adding a fluorophore into the a PCR reaction system, and monitoring the entire PCR process according to the accumulation of fluorescent signal, and finally analyzing a DNA template quantitatively via a standard curve. In one further embodiment, the expression levels of the miRNA and the internal control RNA are determined by the qRT-PCR and expressed as a cycle threshold (Ct) or the actual level of RNA converted from the Ct. As used herein, the technical term “cycle threshold (Ct)” is defined as the number of cycles required for the fluorescent signal to cross the threshold in real-time PCR.

In one embodiment, the internal control RNA is the RNA with stable expression level, which includes, for example, a 18S ribosomal RNA (18S rRNA), a U6B small nuclear RNA (U6B snRNA), and a mature miRNA-16 (miR-16). As used herein, the technical term “RNA with stable expression level” is defined as the RNA whose expression level does not change significantly in the pathogenic process (within 95% confidence interval). In another embodiment, the internal control RNA is the mature miR-16 comprising a nucleotide sequence of SEQ ID NO: 3.

In one embodiment, the relative expression level of the candidate miRNA is the expression level of the candidate miRNA corrected by the expression level of the internal control RNA. In one further embodiment, the relative expression level of the candidate miRNA is calculated by the Formula I:

The relative expression level of the miRNA=2^{-ΔCt} (Formula I)

Wherein the “ΔCt” is obtained by subtracting the qRT-PCR averaged Ct value of the internal control RNA from the qRT-PCR averaged Ct value of miRNA of interest which as the biomarker of atherosclerosis, wherein the internal control RNA is the RNA with stable expression level. The obtained value is the ΔCt, which is expressed as the following formula:

ΔCt=(Ct−Ct_{internal control RNA})

wherein the Ct is defined as the Ct value of the candidate miRNA, and the Ct_{internal control} RNA is defined as the Ct value of the internal control RNA. The ΔCt is the difference between the two Ct values, which is defined as the ΔCt value of the candidate miRNA.

The technical term “prediction model” recited herein is referred to as a linear prediction model established by the one or more risk factors of disease, so as to predict if the disease will happen in the future. Statistically, the linear prediction model could be established by a regression analysis, and a logistic regression is preferred. In one preferred embodiment, the prediction model may be established as a linear prediction model by using the one or more risk factors of the disease together with the expression levels of the one or more candidate miRNAs. In certain embodiments, the risk factors of the disease may be selected from, but not limited to, the group consisting of age, gender, blood pressure, fasting blood glucose level, total cholesterol level and triglyceride level in blood. The one or more candidate miRNAs could be selected from, but not limited to, the group consisting of the relative expression levels of the mature miRNA-221 and the relative expression levels of the mature miRNA-21. As to other available miRNAs, please refer to the website described in the paper by Griffiths-Jones S et al. (Griffiths-Jones S et al., Nucleic Acids Res., 2008; 36: D154-D158). The website used as a reference in the present invention.

The technical term “prediction probability” recited herein is defined as generating a linear combination from the model mentioned above for predicting the probability of an individual developing the disease in the future. The prediction probability is calculated as follows:

p=1−1/[1+E(linear combination)]

wherein, the E (linear combination) is defined as an exponential function of the linear combination which is obtained from the correlation between the risk factors and the disease, and the p is the prediction probability.

The technical term “sensitivity” recited herein is referred to as the percentage of the individuals who actually have the disease and also confirmed by the detection.

The technical term “specificity” used herein is referred to the percentage of the individuals who actually do not have the disease and also confirmed by the detection.

The present invention also includes a method for in vitro diagnosis of atherosclerosis, which may be further applied to the diseases selected from a group consisting of stroke, myocardial infarction, acute coronary syndrome, and other diseases caused by atherosclerosis. The diagnostic method mentioned above includes the following steps: (a) obtaining a sample from a subject, wherein the sample is blood, tissue or body fluid, and blood is preferred; (b) determining expression levels of one or more miRNAs (microRNAs) as atherosclerosis biomarkers and expression level of an internal control RNA; (c) computing relative expression levels of the one or more miRNAs as atherosclerosis biomarkers; (d) computing a prediction model by using one or more variables, wherein the variables include relative expression levels of the one or more miRNAs as atherosclerosis biomarkers and one or more risk factors of atherosclerosis; and (e) computing a disease risk probability by the prediction model. The subject is diagnosed as atherosclerosis if the disease risk probability is greater than 0.5.

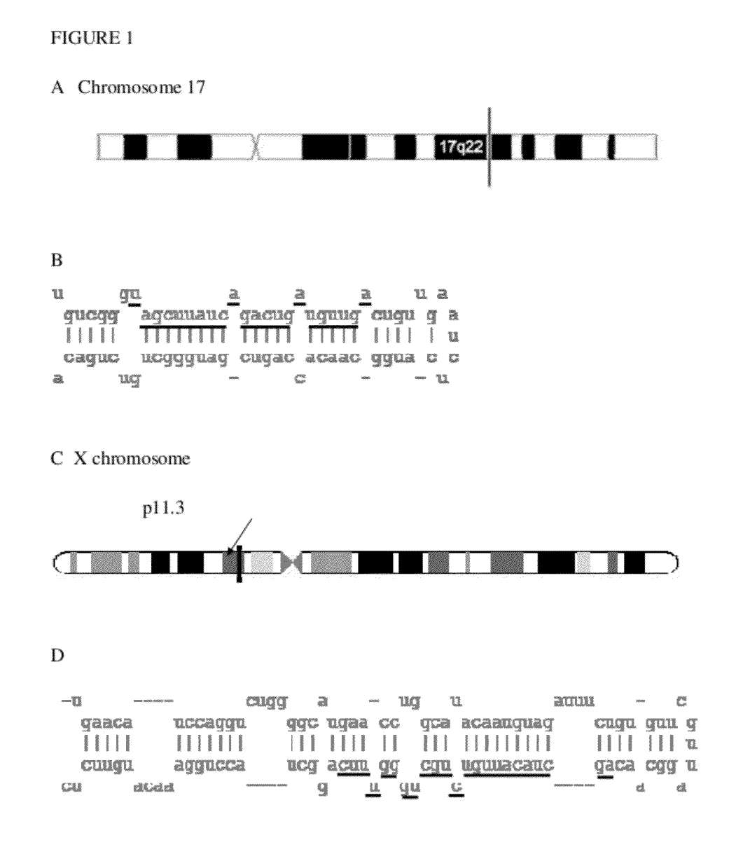

In the step (b), the one or more miRNAs selected as atherosclerosis biomarkers are obtained from the method for selecting a miRNA for use as a disease diagnostic biomarker. In certain embodiments, the one or more miRNAs selected as the biomarkers of atherosclerosis may be selected from the miRNAs whose expression levels are significantly different (ie. much higher or lower) between a patient with atherosclerosis or stroke and a normal person without atherosclerosis or stroke. In one embodiment, the one or more miRNAs selected as the diagnostic biomarkers of atherosclerosis are selected from the group consisting of pri-miR-21, pre-miR-21, mature miRNA-21, pri-miR-221, pre-miR-221 and mature miRNA-221 (FIG. 1). In one further embodiment, the one or more miRNAs selected as the diagnostic biomarkers of atherosclerosis are the mature miRNA-21 and the mature miRNA-221, wherein the mature miRNA-21 comprises a nucleotide sequence of SEQ ID NO: 1 and the mature miRNA-221 comprises a nucleotide sequence of SEQ ID NO: 2.

The one or more miRNAs selected from the group composed of the pri-miR-21, the pre-miR-21, the mature miRNA-21, the pri-miR-221, pre-miR-221, and the mature miRNA-221 may be used as the reference parameter for evaluating effect of an atherosclerosis treatment and screening a drug against atherosclerosis. In one embodiment, the mature miRNA-21 and the mature miRNA-221 are used as the reference parameters when evaluating the effect of an atherosclerosis treatment and screening a drug or drugs against atherosclerosis.

The present invention further provides a kit for diagnosing atherosclerosis and/or predicting diseases caused by atherosclerosis in vitro, which comprising: (a) a mature miRNA-21 quantitative kit includes pairs of nucleotide primers and detection reagents for determining expression levels of mature miRNA-21 and mature miRNA-16; (b) a mature miRNA-221 quantitative kit includes pairs of nucleotide primers and detection reagents for determining expression levels of mature miRNA-221 and mature miRNA-16; and (c) a programmable object, which is provided for inputting the expression levels of the mature miRNA-21, the mature miRNA-221 and the mature miRNA-16 to perform the calculation process of the above-mentioned method for in vitro diagnosis of atherosclerosis.

The technical term “programmable object” mentioned in the present invention is referred to as an object having a program instruction, and when the programmable object is stored in the computer readable medium, it provides data processing capability for the computer to point out, complete or accomplish a specific function, job or result.

In one further embodiment, the kit could be applied as the reference parameter for evaluating effect of a treatment for atherosclerosis and screening a drug or drugs against atherosclerosis.

The miRNA-221 and miRNA-21 are used as the biomarkers in the detection kit of the present invention, and the advantages includes: (1) it is easy to diagnose the disease when used with qRT-PCR; (2) it is an accurate method with both high sensitivity (detecting the individuals with atherosclerosis effectively) and high specificity (detecting the individuals without atherosclerosis effectively); (3) it has a high value in clinical application, especially for diagnosing atherosclerosis and predicting the risk of stroke and myocardial infarction; (4) it can be used in predicting the progress of atherosclerosis; and (5) it is easy to get the assay samples (such as serum or plasma).

### EXAMPLES

The examples below are non-limiting and are merely representative of various aspects and features of the present invention.

### Example 1

Detection of the Levels of miRNA in Blood and the Biochemical Indicator

384 subjects were included in the developing process of the present invention, which included 167 subjects with ischemic stroke, 34 atherosclerosis subjects with a carotid plaque score ≧3 and 181 healthy controls with plaque score ≦1. These three types of subjects represented three levels of severity in cerebrovascular diseases. The plaque index was used as an index for the severity of atherosclerosis, and the higher the plaque index was the more severe atherosclerosis. According to the document published by Sutton-Tyrrell et al. (Sutton-Tyrrell K, et al., Stroke. 1998; 29: 1116-21), plaque index was calculated by summing up the plaque grades of five segments of bilateral carotid artery (proximal and distal common carotid artery (CCA), bifurcation (Bif), internal carotid artery, and extracranial carotid artery). The level of miRNA and the biochemical indicator were measured from blood, which included, for example, the level of the mature miRNA-21, the level of the mature miRNA-221, the level of the mature miRNA-16, the fasting blood glucose, the total cholesterol, and the triglyceride. The subjects had provided their consents before the study was conducted. The measurements of the fasting blood glucose, the total cholesterol, and the triglyceride were performed according to the standard assays commonly used in the hospital setting. The level of the miRNA in blood was measured as follows: firstly, approximately 6 mL venous blood was collected from an antecubital fossa and placed in a serum separator tube. The blood was centrifuged at 3,000 rpm for 10 min and then serum was obtained and aliquoted into a 1.5 mL microcentrifuge tube. Total RNA in serum including miRNA was separated by using a MasterPure™ RNA (Epicentre) purification kit according to the manufacturer's protocol. The RNA was stored at a temperature of −80° C. until use.

The level of the miRNA was determined by the quantitative real-time RT-PCR. MicroRNA's quantitative assay for the mature miRNA-21, the mature miRNA-221, or the mature miRNA-16 was quantified by a TaqMan® MicroRNA Assay containing specific primers for miR-21 (Applied Biosystems, assay ID: 000397), miRNA-221 (Applied Biosystems, assay ID: 000524) and miRNA-16 (Applied Biosystems, assay, ID: 000391) respectively. The RT-reaction was used a reverse transcription kit (TaqMan® MicroRNA Reverse Transcription Kit; 4366596), and then reverse transcribed according to the following conditions:

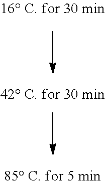

The quantitative PCR was performed as follows: mixing the product of RT with 1 μL of 20× TaqMan MicroRNA Assay reagent and 10 μL of TaqMan 2× Universal PCR Master Mix reagent, and the resulting mixture was used in quantitative PCR. The above mentioned mixture contained the primers and the probes of the assay kit. The condition for quantitative PCR was as follows:

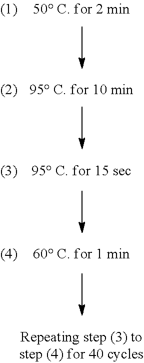

The result of RT-PCR was analyzed by ABI 7900 HT Fast Real-Time PCR System. The relative difference was quantified by the method of ΔCt.

### Example 2

The miRNA-21 and miRNA-221 expression levels were standardized to miRNA-16 expression level to get the relative expression levels. The relative expression of miRNAs was quantified by log 2^{-ΔCt}. If a serum miRNA in one sample had a Ct value greater than 35, the miRNA concentration was considered undetectable in this sample and was coded as missing in the subsequent analysis.

According to one aspect of the embodiment, the data of the expression levels of a set of miRNA-221 and miRNA-21 was determined by ABI 7900 HT Real-Time PCR system, wherein the miRNA-16 was used as the internal control. The purpose of the aspect mentioned above was to determine if there was significant difference of the expression levels of miRNA-221 and miRNA-21 between the subjects with and without atherosclerosis (Table 1).

The Ct values of miR-21 and miR-221 were significantly different among these three study groups (ANOVA p value<0.0001). The mean Ct value of miR-21 was highest in the healthy controls, followed by atherosclerosis subjects and the stroke patients. There were significant differences of mean Ct of miR-21 in pairwise comparisons between atherosclerosis subjects and controls (post hoc p=0.0006) and between stoke patients and controls (post hoc p<0.0001). On the contrary, the mean Ct value of miR-221 was lowest in the healthy subjects, followed by atherosclerosis subjects and then the stroke patients. The difference of mean Ct value of miR-21 between stroke patients and controls was significant (post hoc p<0.0001), but not significant between atherosclerosis subjects and controls which could be due to a small sample size in the atherosclerosis group.

TABLE 1
Demographic data and Ct value of serum miRNAs among the healthy
controls, atherosclerosis subjects and stroke patients.

| N (%) or | Healthy | Atherosclerosis | Stroke | Crude |
|---|---|---|---|---|
| mean ± SD | (n = 181) | (n = 34) | (n = 167) | P value^{†} |
| Female | 107 (59.1) | 15 (44.1) | 56 (33.5)^{§} | <0.0001 |
| Age (years) | 56.4 ± 8.8 | 62.9 ± 6.5^{‡} | 67.4 ± 12.8^{§} | <0.0001 |
| Ever smoking | 17 (9.4) | 5 (14.7) | 35 (21.7)^{§} | 0.006 |
| Diabetes | 12 (6.6) | 12 (35.3)^{‡} | 73 (43.7)^{§} | <0.0001 |
| Hypertension | 39 (21.6) | 18 (52.9)^{‡} | 127 (76.1)^{§} | <0.0001 |
| Hyperlipidemia | 45 (25.0) | 16 (47.1)^{‡} | 54 (32.3) | 0.027 |
| MiR-16 Ct value | 25.4 ± 2.0 | 25.2 ± 2.5 | 25.7 ± 2.0 | 0.247 |
| MiR-21 Ct value | 30.2 ± 1.4 | 29.2 ± 1.7^{‡} | 29.4 ± 1.4 ^{§} | <0.0001 |
| MiR-221 Ct value | 31.5 ± 1.7 | 32.0 ± 1.6 | 32.4 ± 1.4 ^{§} | <0.0001 |

^{†}Crude p value was calculated by ANOVA or X² test.
^{‡}Significant difference (p < 0.05) between atherosclerosis subjects and healthy controls by the Tukey's post-hoc test.
^{§}Significant difference (p < 0.05) between stroke patients and healthy controls by the Tukey's post hoc test.

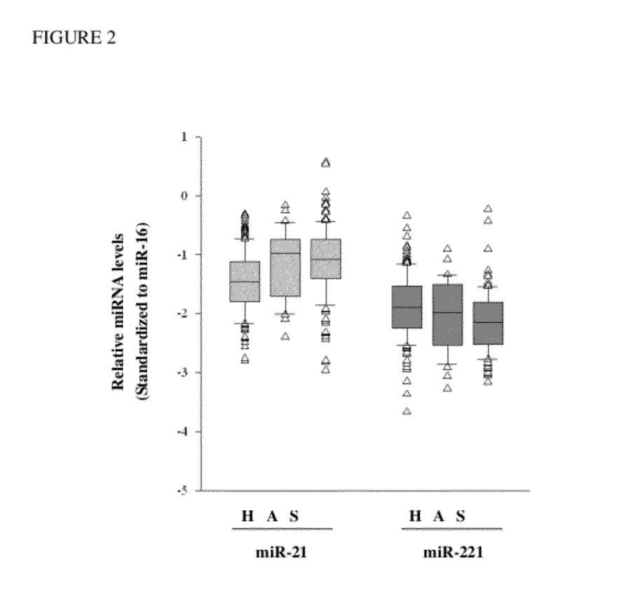

MiR-16 was then used as internal control to standardize the serum levels of miR-21 and miR-221 (log 2^{-ΔCt}). Similar results were found between the analysis of Ct values and relative RNA levels standardized to miR-16. After adjusting for the traditional risk factors, including age, sex, hypertension, diabetes, hyperlipideima and smoking history, standardized miR-21 and miR-221 levels remained to be significantly associated with disease severity (FIG. 2, miR-21: adjusted p=0.0007 for the trend; miR-221: adjusted p=0.0005 for the trend).

The statistical software package used for data analysis was JMP software (version 7.0.1, SAS Institute Inc., Cary, N.C.). A series of multivariable logistic regression models were built to predict the stroke risk. For these analyses, only 167 stroke cases and 181 healthy controls were included. The effect size of each risk factor is shown in Table 2. In addition to known risk factors (age, sex, diabetes and hypertension), subjects with a 2-fold increase of standardized miR-21 level or 2-fold decrease of standardized miR-221 level had approximately 2-fold risk for stroke (OR=1.83 and p value<0.0001 for miR-21; OR=1.99 and p value<0.0001 for miR-221). In addition, there was not any significant interaction between standardized miR-21 and miR-221 (p for interaction=0.11). The regression model suggested that both miRNAs were independent factors to predict ischemic stroke.

TABLE 2
The association of potential risk factors and stroke

| Risk factors | OR (95% CI) | P value |
|---|---|---|
| Age (years) | 1.07 (1.04, 1.11) | 0.0001 |
| Sex (Male) | 3.59 (1.62, 8.27) | 0.002 |
| Ever smoking | 1.13 (0.36, 3.70) | 0.810 |
| Diabetes | 8.83 (3.33, 26.27) | <0.0001 |
| Hypertension | 6.46 (3.03, 14.32) | <0.0001 |
| Hyperlipidemia | 1.30 (0.58, 3.03) | 0.531 |
| Increased miR-21 level | 1.83 (1.43, 2.40) | <0.0001 |
| Decreased miR-221 level | 1.99 (1.55, 2.64) | <0.0001 |

^{†}The p value for interaction term between miR-21 and miR-221 was 0.113.

Different multivariate logistic models were compared to find the best predictive model for stroke. The receiver operating characteristic (ROC) curve and the area under curve (AUC) were used to indicate the predictive capability of these models. AUC values were pairwise compared in different models by using the MedCalc software (Mariakerke, Belgium). The prediction probability of stroke was calculated by the formula II (Chen J Y, et al., Int Heart J 2006; 47: 259-271):

Probability=1−(1/[1+E(regression model)] (Formula II)

A subject with a probability higher than 0.5 was favored to have stroke. The sensitivity and specificity were calculated according to the standard formulas.

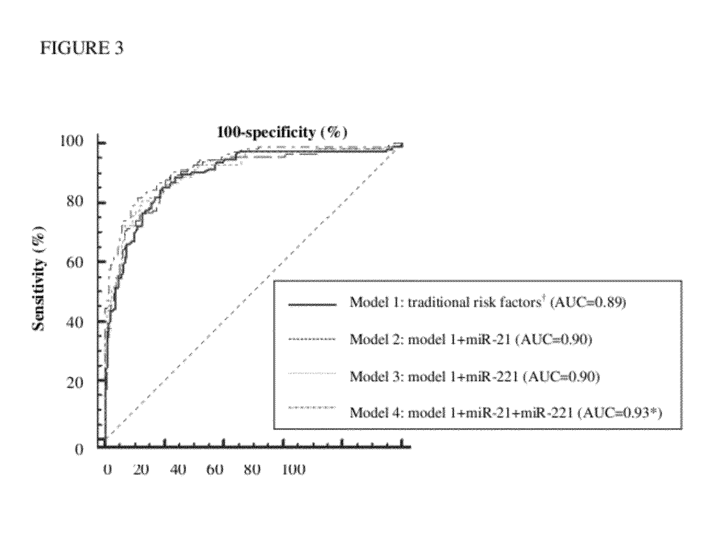

To select the best model for stroke prediction, the significant standardized miRNAs were initially added into the models containing traditional risk factors (model 1: traditional risk factors; model 2: model 1 plus miR-21; model 3: model 1 plus miR-221) one by one. Both miR-21 and miR-221 were then included into the model 1 to test the combined effect of these two miRNAs (model 4: model 1 plus miR-21 and miR-221). The prediction capabilities of the four models are shown in FIG. 3. The ROC curves of the four models (models 1-4) yielded the AUC values of 0.89, 0.90, 0.90 and 0.93, respectively. Accordingly, the model containing two miRNAs and traditional risk factors was the best model because it had the highest AUC value. Pairwise comparison of AUC among the four models showed that model 4 was significantly better than the other three models (p value=0.005 for models 4 vs. 1, p=0.007 for models 4 vs. 2, and p=0.023 for models 4 vs. 3) (FIG. 3).

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A method for in vitro diagnosis of atherosclerosis, comprising:
(a) obtaining a sample from a subject;
(b) determining expression levels of one or more miRNAs (microRNAs) as atherosclerotic biomarkers and an internal control RNA;
(c) computing relative expression levels of the one or more miRNAs as atherosclerosis biomarkers;
(d) computing a prediction model by using one or more variables, wherein the variables include relative expression levels of the one or more miRNAs as atherosclerosis biomarkers and one or more risk factors of atherosclerosis; and
(e) computing a disease risk probability by the prediction model, wherein the subject is diagnosed as atherosclerosis if the disease risk probability is greater than 0.5.

2. The method for in vitro diagnosis of atherosclerosis of claim 1, which is further used for predicting a disease selected from the group consisting of diseases caused by atherosclerosis.

3. The method for in vitro diagnosis of atherosclerosis of claim 2, wherein the diseases caused by atherosclerosis include stroke, myocardial infarction and acute coronary syndrome.

4. The method for in vitro diagnosis of atherosclerosis of claim 1, wherein the sample is blood or tissue.

5. The method for in vitro diagnosis of atherosclerosis of claim 1, wherein the one or more miRNAs as atherosclerosis biomarkers are obtained from a method for selecting a miRNA for use as a disease diagnostic biomarker, which comprising:
(a) obtaining samples from subjects, wherein the subjects are composed of people suffering from the disease and people not suffering from the disease;
(b) determining expression levels of candidate miRNAs and an internal control RNA in the samples;
(c) computing relative expression levels of the candidate miRNAs;
(d) computing a prediction model with one or more variables, wherein the variables include relative expression levels of one or more candidate miRNAs and one or more risk factors of the disease; and
(e) computing a disease risk probability, sensitivity and specificity by the prediction model,
wherein the one or more candidate miRNAs with the highest sensitivity and the highest specificity are selected to be the disease diagnosis biomarker.

6. The method for in vitro diagnosis of atherosclerosis of claim 5, wherein the risk factors are risk factors of arteriosclerosis.

7. The method for in vitro diagnosis of atherosclerosis of claim 1, wherein the one or more miRNAs are selected from the group consisting of pri-miR-21, pre-miR-21, mature miRNA-21, pri-miR-221, pre-miR-221 and mature miRNA-221.

8. The method for in vitro diagnosis of atherosclerosis of claim 7, wherein the one or more miRNAs are used as a reference parameter for evaluating effect of an atherosclerosis treatment and screening a drug against atherosclerosis.

9. The method for in vitro diagnosis of atherosclerosis of claim 7, wherein the mature miRNA-21 and mature miRNA-221 have significantly different expression level between an atherosclerosis or stroke patient and normal healthy subjects.

10. The method for in vitro diagnosis of atherosclerosis of claim 7, wherein the mature miRNA-21 comprises a nucleotide sequence of SEQ ID NO: 1.

11. The method for in vitro diagnosis of atherosclerosis of claim 7, wherein the mature miRNA-221 comprises a nucleotide sequence of SEQ ID NO: 2.

12. The method for in vitro diagnosis of atherosclerosis of claim 1, wherein the internal control RNA is a RNA with stable expression.

13. The method for in vitro diagnosis of atherosclerosis of claim 12, wherein the RNA with stable expression is mature miRNA-16 comprising a nucleotide sequence of SEQ ID NO: 3, 18S rRNA or U6B snRNA.

14. The method for in vitro diagnosis of atherosclerosis of claim 12, wherein the RNA with stable expression is mature miRNA-16 comprising a nucleotide sequence of SEQ ID NO: 3.

15. The method for in vitro diagnosis of atherosclerosis of claim 1, wherein the expression levels of one or more miRNAs and internal control RNA are determined by quantitative real-time RT-PCR and expressed as a cycle threshold (Ct).

16. The method for in vitro diagnosis of atherosclerosis of claim 1, wherein the relative expression levels of the one or more miRNAs are calculated by the formula I:
The relative expression level of miRNA=2^{-ΔCt} (formula I);
wherein the ΔCt is obtained by substracting qRT-PCR averaged Ct value of an internal control RNA from qRT-PCR averaged Ct value of a miRNA as the biomarker of atherosclerosis.

17. The method for in vitro diagnosis of atherosclerosis of claim 1, wherein the one or more risk factors of atherosclerosis is selected from the group consisting of age, gender, blood pressure, fasting blood glucose level, total cholesterol level and triglyceride level in blood.

18. A kit for diagnosing atherosclerosis and/or predicting diseases caused by atherosclerosis in vitro, which comprising: (a) a mature miRNA-21 quantitative kit includes pairs of nucleotide primers and detection reagents for determining expression levels of mature miRNA-21 and mature miRNA-16, (b) a mature miRNA-221 quantitative kit includes pairs of nucleotide primers and detection reagents for determining expression levels of mature miRNA-221 and mature miRNA-16, and (c) a programmable object, which is provided for inputting the expression levels of the mature miRNA-21, the mature miRNA-221 and the mature miRNA-16 to perform the method for in vitro diagnosis of claim 1.
